(19) United States
(12) Patent Application Publication
(10) Pub. No.: US 2023/0089727 A1
(43) Pub. Date: Mar. 23, 2023

(54) PLASMA PROTEOMICS PROFILING BY AUTOMATED ITERATIVE TANDEM MASS SPECTROMETRY

(71) Applicant: Regeneron Pharmaceuticals, Inc., Tarrytown, NY (US)

(72) Inventors: Xiaoxiao Huang, Briarcliff Manor, NY (US); Yu Huang, Ossining, NY (US); Xiaojing Zheng, Gaithersburg, MD (US); Ning Li, New Canaan, CT (US)

(21) Appl. No.: 17/946,757

(22) Filed: Sep. 16, 2022

Related U.S. Application Data
(60) Provisional application No. 63/245,532, filed on Sep. 17, 2021.

(51) Int. Cl. G01N 33/68; G01N 30/72; G01N 30/88; G01N 30/86

(52) U.S. Cl. CPC G01N 33/6848; G01N 30/7233; G01N 30/88; G01N 30/8631; G01N 2030/027

(57) ABSTRACT

The present invention generally pertains to methods of characterizing at least one protein of interest in a biological sample. In particular, the present invention pertains to the use of automated iterative tandem mass spectrometry (AIMS) to identify, quantify and characterize at least one protein of interest and/or biomarker from a biological sample such as plasma.

### CROSS-REFERENCE TO RELATED APPLICATIONS

This application claims priority to and the benefit of U.S. Provisional Patent Application No. 63/245,532, filed Sep. 17, 2021 which is herein incorporated by reference.

### FIELD

This application relates to methods for characterization of proteins of interest in a biological sample.

### BACKGROUND

Blood plays a central role in facilitating diverse biological processes. Whole blood is an easily accessible and minimally invasive tissue that affords a significant opportunity to study human biology and to detect signs of disease using biomarkers. Plasma, the liquid component of whole blood, can be obtained after centrifugation of whole blood in the presence of anti-coagulants. This isolation eliminates cellular material and leaves cell-free components available for detailed characterization.

Plasma is a challenging biological matrix, due to both a large dynamic range in protein expression and the capabilities of state-of-the-art analytical methods. The high-throughput and sensitive nature of mass spectrometry (MS) makes it an attractive method for conducting plasma proteomics studies. However, because of the large dynamic range of plasma protein concentrations, conventional MS methods may require enrichment steps before plasma proteins can be analyzed, which leads to loss of protein and complicated preparation steps.

Therefore, it will be appreciated that a need exists for methods and systems to sensitively analyze proteins in a complex biological sample, for example plasma, using a simple preparation method that does not require enrichment steps.

### SUMMARY

A method has been developed for plasma proteomics profiling using automated iterative tandem mass spectrometry (AIMS). The method may include sample preparation steps of acquiring plasma from a subject, diluting the plasma in lysis buffer, contacting the sample of the diluted plasma to at least one protein reducing agent, contacting the sample to at least one protein alkylating agent, contacting the sample to at least one digestive enzyme, subjecting the sample to peptide cleanup, and concentrating the peptides of the sample. The method may include analysis steps of subjecting the sample to liquid chromatography (LC) to obtain chromatographic elution peaks, further subjecting the chromatography-separated sample to tandem mass spectrometry (MS/MS) using data-dependent acquisition (DDA), automatically selecting precursor ions from the mass spectrum scan to add to an automatic exclusion set, and repeating this LC-MS/MS analysis while excluding precursor ions in the automatic exclusion set for a predetermined number of cycles. The use of automated iterative MS/MS (AIMS) as described allows for more comprehensive detection of lower-abundance precursor ions, as high-abundance precursor ions are excluded from redundant mass spectrum scans after being added to an automatic exclusion set, and thus more sensitive and accurate proteomics profiling.

This disclosure provides a method for characterizing at least one protein of interest in a biological sample. In some exemplary embodiments, the method comprises (a) subjecting a biological sample to a chromatography column to obtain a chromatographic elution peak; (b) performing a tandem mass spectrometry analysis by performing a data-dependent acquisition cycle across the chromatographic elution peak of (a), wherein the cycle includes: (i) obtaining a mass spectrum scan; (ii) selecting a plurality of precursor ions from the obtained mass spectrum scan as an automatic exclusion set; and (iii) obtaining a second mass spectrum scan after excluding the plurality of precursor ions set in the automatic exclusion set; and (c) characterizing the at least one protein of interest after the acquisition cycle is run for a predetermined number of times.

In one aspect, the predetermined number of cycles is one, two, three, four, or more cycles. In another aspect, a mass error tolerance for selecting a precursor ion for an automatic exclusion set is at about 15 ppm. In yet another aspect, a retention time tolerance for selecting a precursor ion for an automatic exclusion set is from about −0.2 minutes to about +0.4 minutes.

In one aspect, the automatic exclusion set also includes at least one background ion. In another aspect, the automatic exclusion set includes at least one additional precursor ion not from the acquired mass spectrum scan. In yet another aspect, precursor ions from the acquired mass spectrum are not added to the automatic exclusion set if they fall below a predetermined intensity threshold.

In one aspect, the sample preparation includes direct digestion. In a specific aspect, direct digestion comprises contacting said sample to trypsin and LysC.

In one aspect, the chromatography step comprises reverse phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, the mass spectrometer is an electrospray ionization mass spectrometer, nano-electrospray ionization mass spectrometer, or a quadrupole time-of-flight mass spectrometer, wherein the mass spectrometer is coupled to a liquid chromatography system.

In one aspect, the biological sample is a human sample. In another aspect, the biological sample is plasma. In yet another aspect, the at least one protein of interest is a biomarker.

Additionally, this disclosure provides a method for characterizing at least one biomarker in human plasma. In some exemplary embodiments, the method comprises (a) diluting about 5 μL of human plasma in lysis buffer; (b) taking a sample of said diluted human plasma comprising about 100 μg of plasma protein; (c) contacting said sample to at least one reduction agent and at least one alkylation agent; (d) contacting said sample from (c) to trypsin and LysC under digestive conditions to form a digested peptide sample; (e) subjecting said digested peptide sample to peptide cleanup; (f) subjecting said digested peptide sample of (e) to an overnight concentration step to form a concentrated peptide sample; (g) subjecting said concentrated peptide sample to a chromatography column to obtain a chromatographic elution peak; (h) performing a tandem mass spectrometry analysis by performing a data-dependent acquisition cycle across the chromatographic elution peak of (a), wherein the cycle includes: (i) obtaining a mass spectrum scan; (ii) selecting a plurality of precursor ions from the obtained mass spectrum scan as an automatic exclusion set; and (iii) obtaining a second mass spectrum scan after excluding the plurality of precursor ions set in the automatic exclusion set; and (c) characterizing the at least one biomarker after the acquisition cycle is run for at least three times.

These, and other, aspects of the present invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 shows a workflow of sample preparation according to an exemplary embodiment.

FIG. 2 shows a workflow of automated iterative MS/MS according to an exemplary embodiment.

FIG. 3 shows a data acquisition file for automated iterative MS/MS according to an exemplary embodiment.

FIG. 4 shows unique peptides identified by each run of a regular data-dependent acquisition (DDA) method compared to an iterative DDA method according to an exemplary embodiment.

FIG. 5 shows protein groups identified by each run of a regular DDA method compared to an iterative DDA method according to an exemplary embodiment.

FIG. 6A shows stable retention times of 15 peptides over the course of 60 LC-MS injections according to an exemplary embodiment. FIG. 6B shows stable peak areas of 15 peptides over the course of 60 LC-MS injections according to an exemplary embodiment.

FIG. 7 shows a quantitation of known biomarkers in 14 individual patient samples using automated iterative MS/MS according to an exemplary embodiment.

FIG. 8 shows a quantitation of serum amyloid A 4 (SAA4) in healthy patients and patients with irritable bowel syndrome (IBS) using automated iterative MS/MS according to an exemplary embodiment.

FIG. 9 shows a quantitation of the most abundant apolipoproteins in healthy patients and patients with nonalcoholic steatohepatitis (NASH) using automated iterative MS/MS according to an exemplary embodiment.

FIG. 10 shows a quantitation of low-abundance apolipoproteins in healthy patients and patients with NASH using automated iterative MS/MS according to an exemplary embodiment.

### DETAILED DESCRIPTION

The need for more clinically relevant biological insights is driving an increase in the number of mass spectrometry (MS)-based proteomic studies. Plasma is an attractive biological material for clinical study because blood plays a central role in facilitating diverse biological processes and is easily accessible with minimally invasive procedures. Therefore, plasma proteomics holds great promise for the future of biomarker discovery, as well as in vitro diagnostics.

A major challenge of plasma proteomics is the wide dynamic range of protein abundance, spanning 10 orders of magnitude between the most and least abundant plasma proteins. Conventional methods require extensive preparation of plasma samples prior to analysis in order to reduce the dynamic range of plasma proteins. This can include, for example, plasma depletion and protein precipitation, which result in loss of sample and a distortion of the relative abundance of plasma proteins. Each additional preparation step also adds a cost in time and resources to plasma proteomics analysis. However, forgoing these steps and using an unenriched sample may result in inaccurate plasma protein characterization by MS.

For example, traditional proteomics methods such as data dependent acquisition (DDA) only fragment the most abundant precursors in a sample, and as a result may miss low abundance ions. DDA methods inherently bear some limitations for identification of proteins with large dynamic of abundance ranges. The limitation stems from the finite number of precursors chosen for MS2 scan in each MS1 scan, where “MS1” and “MS2” indicate the first and second mass analysis in tandem mass spectrometry respectively. Due to the actual duty cycle of the MS2 scans, some complex MS1 scans have very few precursor ions selection for MS2, leaving a large number of precursors not selected for MS2 identification. Besides, the fluctuation of low-level ions in a complex MS1 scan might result in stochastic precursor selection, leading to inconsistent or complementary identifications from each replication injection.

Alternatively, LC-MS/MS with data independent acquisition (DIA), particularly the SWATH method, has been shown as a promising strategy to simultaneously identify and quantitate proteins (Heissel et al., 2018, Protein Expr Purif, 147:69-77; Walker et al., 2017, MAbs, 9:654-663). Although the DIA-based method is able to generate a multiplexed MS2 from a window of precursor ions capturing MS2 fragments from theoretically all precursors, it still faces a small window dynamic range interference issue. In addition, a big hurdle is in data processing for effective and reliable protein identification, as well as the prerequisite of constructing a protein library. All of these challenges limit the application of DIA in plasma proteomics. DDA, therefore, serves as an optimal method for identification of peptides and proteins for LC-MS/MS.

The recent development of a new MS acquisition method for deep proteomics, iterative precursor ion exclusion, has been shown to increase the depth of traditional tandem MS (Zhang, 2012, J Am Soc Mass Spectrom, 23:1400-1407; Wu et al., 2012, Proteomics Clin Appl, 6:304-308; Wang et al., 2008, Anal Chem, 80:4696-4710; Zhou, et al., 2015, J Proteomics Bioinform, 8:260-265; Huang et al., 2021, J Pharm Biomed Anal, 200:114069). In an iterative fashion, the identification of new and unique peptide precursor ions would increase when other precursors that had already been selected for MS/MS fragmentation are excluded from further analysis. Iterative MS/MS is a straightforward acquisition method that could achieve identification and relative quantification of proteins with a wide range of abundances without the need for enrichment. An iterative MS/MS method has not yet been optimized for use in human plasma proteomics, a uniquely challenging type of analysis.

To meet the challenges of plasma proteomics analysis, described herein is a simple, robust and unbiased strategy utilizing an automated precursor ion exclusion acquisition method, or automated iterative MS/MS (AIMS). This AIMS approach can use directly digested samples without requiring any enrichment of the samples. With this AIMS strategy, peptide precursor ions from a complex sample with a wide dynamic range, such as plasma, can be picked up for MS/MS identification in iterative replicates. Therefore, this approach is able to achieve sensitive protein identification and characterization compared to a normal DDA method, with a simpler sample preparation process than conventional methods. Compared to conventional methods, this novel method is capable of identifying a greater number of unique peptides and identifying a greater number of unique proteins while using a simpler sample preparation process that does not require plasma depletion or protein precipitation.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications, which are hereby incorporated by reference: U.S. Ser. No. 12/823,838, filed Jun. 25, 2010; U.S. Ser. No. 13/488,628, filed Jun. 5, 2012; U.S. Ser. No. 14/031,075, filed Sep. 19, 2013; U.S. Ser. No. 14/808,171, filed Jul. 24, 2015; U.S. Ser. No. 15/713,574, filed Sep. 22, 2017; U.S. Ser. No. 15/713,569, field Sep. 22, 2017; U.S. Ser. No. 15/386,453, filed Dec. 21, 2016; U.S. Ser. No. 15/386,443, filed Dec. 21, 2016; U.S. Ser. No. 15/223,43 filed Jul. 29, 2016; and U.S. Ser. No. 15/814,095, filed Nov. 15, 2017.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

In some exemplary embodiments, the protein of interest can be a biomarker. As used herein, the term “biomarker” refers to any substance, structure, or process that can be measured in the body or its products and influence or predict the incidence of outcome or disease. A biomarker may be, for example, a protein that has higher or lower expression as a reflection of the disease state of an individual. A biomarker may also be a species that changes in characteristics in response to a therapy or treatment, and may be measured as an indicator of the effects of said therapy or treatment. In some exemplary embodiments, a biomarker may be a protein that is affected by a disease state in a way that is measurable using mass spectrometry. In some specific exemplary embodiments, a biomarker may be an apolipoprotein. In other specific exemplary embodiments, a biomarker may be a serum amyloid A (SAA) protein.

As used herein, “sample” can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, viral inactivation, or filtration.

In some exemplary embodiments, the sample is a biological sample. As used here, the term “biological sample” refers to a sample taken from a living organism, for example a human or a non-human mammal. A biological sample may comprise, for example, whole blood, plasma, serum, saliva, tears, semen, cheek tissue, organ tissue, urine, feces, skin, or hair. In some exemplary embodiments, the sample may include at least one biomarker. In some exemplary embodiments, the sample may include at least one protein of interest. Analysis of a biological sample presents specific challenges, for example, the variety of the components of the sample, the high concentration of components of the sample, or the wide dynamic range of components of the sample. The present disclosure provides methods for analyzing a biological sample, comprising, for example, a simple sample preparation followed by automated iterative MS/MS.

In some exemplary embodiments, the sample including the protein of interest can be prepared prior to automated iterative MS/MS analysis. Preparation steps can include dilution, alkylation, reduction, denaturation, digestion, peptide cleanup, and/or concentration.

In some exemplary embodiments, about 5 μL of plasma is obtained from a subject for AIMS analysis. Plasma may be pooled from multiple individuals, or may be from only one individual. Plasma may be diluted, and a portion or aliquot of the diluted plasma may be taken for further analysis. Plasma may be diluted in, for example, lysis buffer. A sample of diluted plasma may include about 100 μg of plasma protein. In some exemplary embodiments, the iterative method of the invention includes three LC-MS/MS cycles, each of which analyzes about 30 μg of plasma protein. AIMS may include more than three cycles, for example, four, five, or six cycles. In this case, a greater amount of starting protein may be desired, for example, about 150 μg of plasma protein, about 200 μg of plasma protein, or more. While plasma is used as an example of a complex biological sample that can be analyzed using the method of the present invention, it should be understood that any complex sample and any biological sample can be used in the method of the invention.

As used herein, the term “protein alkylating agent” or “alkylation agent” refers to an agent used for alkylating certain free amino acid residues in a protein. Non-limiting examples of protein alkylating agents are iodoacetamide (IOA/IAA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” or “reduction agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

In some exemplary embodiments, a sample may be concentrated prior to LC-MS/MS analysis. Concentration may comprise, for example, subjecting a sample to vacuum concentration for several hours, or overnight. A device useful for a concentration step may be, for example, a SpeedVac® vacuum concentrator.

In some exemplary embodiments, sample preparation does not include a plasma depletion step. In some exemplary embodiments, sample preparation does not include a protein precipitation step.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some aspects, the sample containing the at least one biomarker or at least one protein of interest can be subjected to any one of the aforementioned chromatographic methods or a combination thereof.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS², can be performed by first selecting and isolating a precursor ion (MS′), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary embodiments, automated iterative MS/MS can be performed under native conditions. As used herein, the term “native conditions” can include performing mass spectrometry under conditions that preserve non-covalent interactions in an analyte. For detailed review on native MS, refer to the review: Elisabetta Boeri Erba & Carlo Pe-tosa, The emerging role of native mass spectrometry in characterizing the structure and dynamics of macromolecular complexes, 24 PROTEIN SCIENCE 1176-1192 (2015).

In some exemplary embodiments, the method of the present invention includes a predetermined number of cycles of tandem mass spectrometry analysis. In one aspect, the number of cycles is one, two, three, four, or more cycles. The number of cycles may be chosen depending on the needs of the user with regards to sample quantity, timing, number of peptide spectrum matches, quantification accuracy, or other needs which can be readily determined by one of ordinary skill in the art.

In some exemplary embodiments, the method of the invention includes setting user-determined mass error tolerance and retention time exclusion tolerance to determine the addition of precursor ions to an automatic exclusion set. The mass error tolerance may be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ppm, or another value according to the needs of the user, which can be readily determined by one of ordinary skill in the art. The retention time exclusion tolerance may be from −0.5 min, −0.4 min, −0.3 min, −0.2 min, −0.1 min, or 0 min to +0.8 min, +0.7 min, +0.6 min, +0.5 min, +0.4 min, +0.3 min, +0.2 min, +0.1 min, or 0 min, or another range according to the needs of the user, which can be readily determined by one of ordinary skill in the art.

In some exemplary embodiments, the method of the invention includes generating an automatic exclusion set that is used to exclude precursor ions for ion fragmentation. In one aspect, precursor ions that are acquired in a mass spectrum scan are added to the automatic exclusion set. In another aspect, the automatic exclusion set also includes background ions. Background ions may be present in all mass spectrum scans and not representative of true peptide products. Background ions can be readily identified by one of ordinary skill in the art. In another aspect, the automatic exclusion set includes at least one additional precursor ion not from the acquired mass spectrum scan. A user may choose to predetermine at least one precursor ion that should not be fragmented even if it has not yet been acquired in a mass spectrum scan. In yet another aspect, precursor ions from the acquired mass spectrum are not added to the automatic exclusion set if they fall below a predetermined intensity threshold. A user may want to repeat a mass spectrum analysis of a precursor ion if a previous acquisition was of low quality or signal intensity.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsfedu/prospector/mshome.htm), Byonic (www.proteinmetrics.com/products/byonic) or Sequest (fields.scripps.edu/sequest).

In some exemplary embodiments, the mass spectrometer is coupled to the chromatography system.

It is understood that the present invention is not limited to any of the aforesaid protein(s), protein(s) of interest, biomarker(s), protein alkylating agent(s), protein denaturing agent(s), protein reducing agent(s), digestive enzyme(s), sample(s), biological sample(s), chromatographic method(s), mass spectrometer(s), mass error tolerance(s), retention time exclusion tolerance(s), database(s), or bioinformatics tool(s), and any protein(s), protein(s) of interest, biomarker(s), protein alkylating agent(s), protein denaturing agent(s), protein reducing agent(s), digestive enzyme(s), sample(s), biological sample(s), chromatographic method(s), mass spectrometer(s), mass error tolerance(s), retention time exclusion tolerance(s), database(s), or bioinformatics tool(s) can be selected by any suitable means.

The present invention will be more fully understood by reference to the following Examples. They should not, however, be construed as limiting the scope of the invention.

### EXAMPLES

### Example 1. Sample Preparation

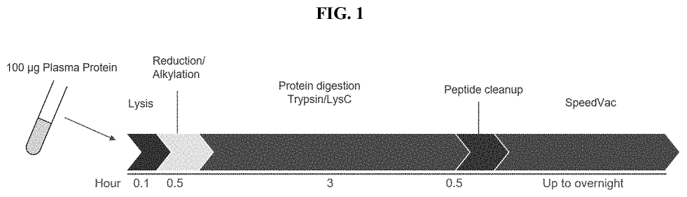

An exemplary process for preparing samples for the AIMS method of the present invention is shown in FIG. 1, using the EasyPep Kit from Thermo Scientific. 5 μL of plasma sample are diluted in lysis buffer. A portion of this diluted sample comprising 100 μg of plasma protein is taken as the sample for subsequent steps. This sample is subjected to a reduction/alkylation step. Then the sample is further subjected to protein digestion using trypsin and LysC. Following digestion, there is a peptide cleanup step. Finally, the sample is subjected to an overnight concentration step, using a SpeedVac® vacuum concentrator, to yield a concentrated, purified peptide sample.

Unlike conventional methods, this method does not require a plasma depletion step, or a protein precipitation and cleaning step. Both steps are commonly used to simplify a plasma sample and enrich for proteins, but result in loss of sample, loss of protein, and additional preparation time. An advantage of the present method is that it allows for the analysis of a complex sample with a high dynamic range of protein concentrations without prior enrichment, and therefore these lossy steps may be avoided, resulting in more accurate identification, characterization and quantitation of proteins.

### Example 2. Automated Iterative MS/MS

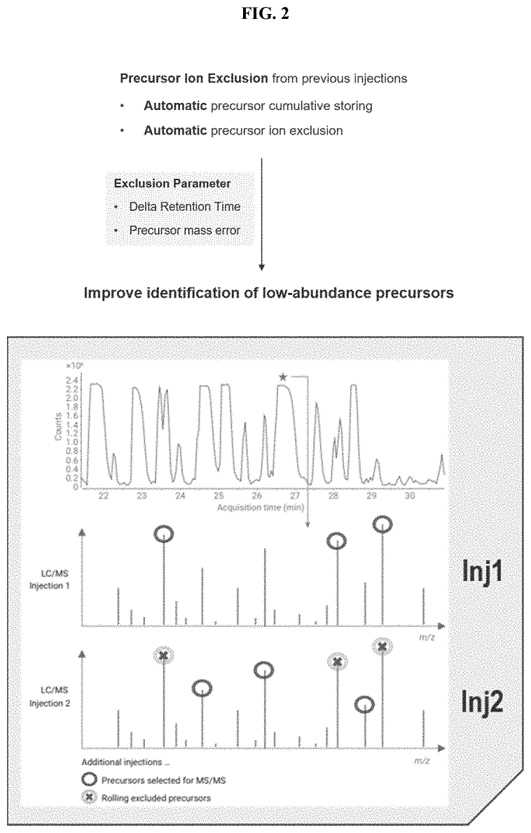

Automated iterative MS/MS (AIMS) with precursor ion exclusion allows for the detection of lower abundance precursors and lower abundance proteins in a sample, as shown in FIG. 2. The identity of precursor ions that are subjected to MS/MS analysis are cumulatively stored. These precursor ions are then automatically excluded from the next MS analysis, allowing for the analysis of less abundant precursor ions. This process is repeated over the course of multiple LC-MS/MS analyses from the same sample. Three injections (runs) were used as the optimal balance of identification sensitivity versus time and resources spent, but the process can instead be repeated four, five, six, or more times if necessary to identify additional peptides.

Exclusion parameters to be customized include retention time tolerance and precursor mass error tolerance. A reasonable amount of variance in retention time and mass must be allowed for, without having too wide an error allowance that would lead to incorrectly excluding precursor ions that had not already been analyzed.

### Example 3. Optimized Method Overview

The sample preparation and analysis steps from Example 1 and Example 2 were optimized for plasma proteomics profiling. The sample starting material was 5 μL of human plasma pooled from multiple subjects. The total plasma protein taken from the starting material was 100 μg. Following the sample preparation step described in Example 1, digested peptides were cleaned and resuspended in 0.1% formic acid (FA) and 3% acetonitrile (ACN).

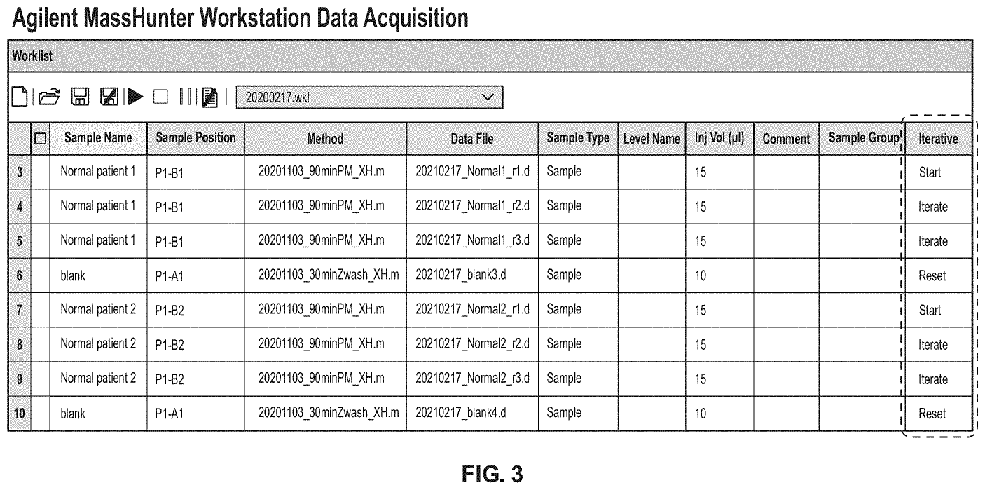

For LC-MS analysis, a mobile phase of 0.1% FA water and ACN was used. Real time mass calibration was performed using split flow of a reference mass from an isocratic pump. The column format used was 2.1 mm×150 mm CSH column. The run time was 90 minutes, plus 1 minute injection time. The injection amount was 30 μg per injection, with 3 iterative DDA injections performed. The sample turnaround time was about 5 minutes plus about 90 minutes per sample, for a total of about 275 minutes. Exemplary data acquisition settings are shown in FIG. 3.

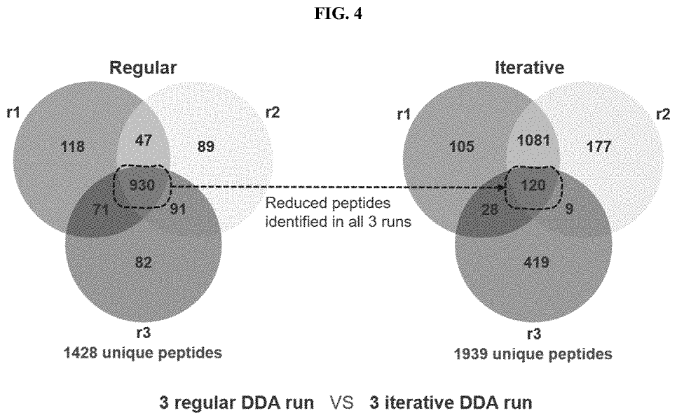

The method of the present invention, using 3 iterative data-dependent acquisition (DDA) MS/MS runs, was compared to 3 regular DDA runs. Using the regular method, 1428 unique peptides were identified, with 930 being identified repeatedly across the 3 runs, as shown in FIG. 4. In contrast, using the iterative method, 1939 unique peptides were identified, with only 120 being identified repeatedly across the 3 runs. This result demonstrates a substantially improved ability of the method of the present invention to reduce redundant identifications of peptides and as a result to identify a far greater number of unique peptides in a complex sample.

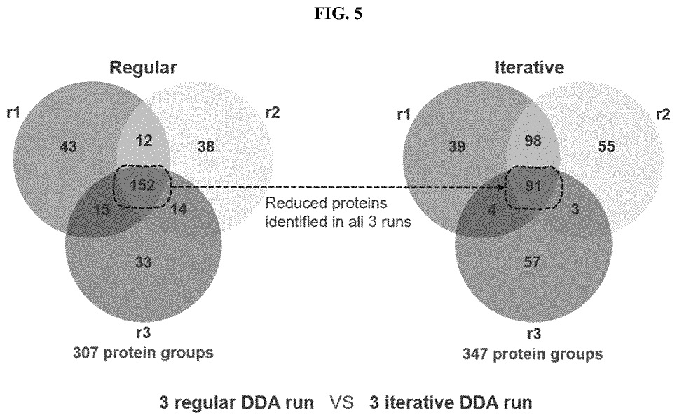

The iterative method was further compared to the regular method in terms of the ability of each method to identify unique protein groups (proteins and variants of those proteins), as shown in FIG. 5. The regular method was able to identify 307 protein groups, with 152 being repeatedly identified across the 3 runs. The iterative method was able to identify 347 protein groups, with only 91 being repeatedly identified across the 3 runs. This result further demonstrates the superiority of the method of the present invention in identifying unique protein groups in a complex sample compared to conventional methods, by effectively reducing redundant identifications between runs.

Protein IDs that were identified in human plasma using the iterative method but not the regular method include P06241-3 Isoform 3 of Tyrosine-protein kinase Fyn, P32189-3 glycerol kinase, Q15643 Thyroid receptor-interacting protein 11, 060241-4 Isoform 4 of Adhesion G protein-coupled receptor B2, Q2TB90-1 Putative hexokinase HKDC1, P42345 Serine/threonine-protein kinase mTOR, Q9NZK5 Adenosine deaminase 2, Q9UK55 Protein Z-dependent protease inhibitor, Q13435 Splicing factor 3b subunit 2, Q2TB90-1 Putative hexokinase HKDC1, Q4ADV7 RAB6A-GEF complex partner protein 1, Q4VNC0 Probable cation-transporting ATPase 13A5, Q502W6-6 Isoform 6 of von Willebrand factor A domain-containing protein 3B, P01877 Immunoglobulin heavy constant alpha 2, P02766 Transthyretin, P10643 Complement component C7, P12111-2 Isoform 2 of Collagen alpha-3(VI) chain, P13942-8 Isoform 8 of Collagen alpha-2(XI) chain, P16989-1 Y-box-binding protein 3, P28562 Dual specificity protein phosphatase 1, P29374-1 AT-rich interactive domain-containing protein 4A, P32189-3 glycerol kinase, P35611-3 Isoform 3 of Alpha-adducin, P29374-1 AT-rich interactive domain-containing protein 4A, and Q9P2E2-1 Kinesin-like protein KIF17. Several of these proteins are potentially of interest as disease biomarkers, suggesting that the method of the present invention may have a substantial advantage when applied to biomarker analysis compared to the conventional method.

### Example 5. Instrument Robustness Test

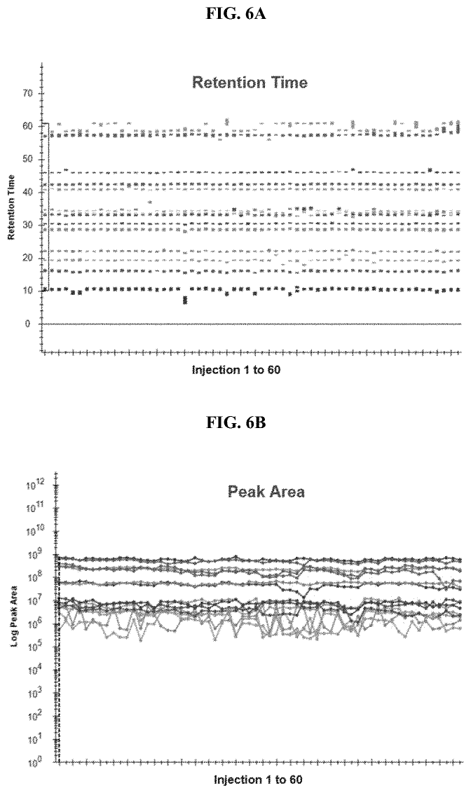

The robustness of the method of the present invention was evaluated. Twenty human plasma samples were run in 60 injections over the course of a week. Four proteins represented by 15 peptides with a range of retention times and abundance were tracked across the runs. The overall retention time and peak area of the proteins and peptides were consistent over time, as shown in FIG. 6A and FIG. 6B. The protein identification level remained the same over time. These results demonstrate that the method of the present invention is robust for repeated use and can be used to reliably compare peptides and proteins across samples and across runs.

### Example 6. Biomarker Analysis

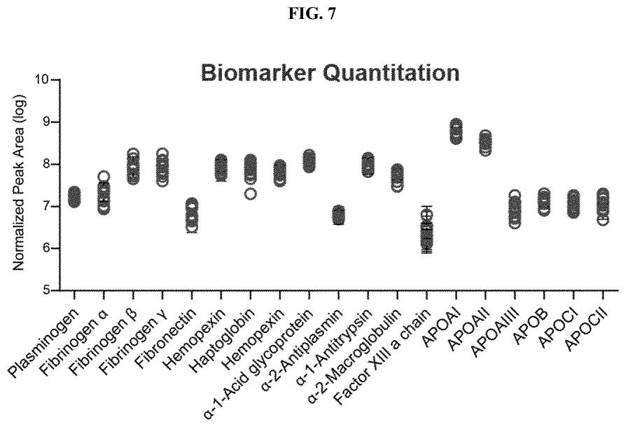

The FDA has cleared or approved multiple tests for proteins in plasma or serum as biomarkers of health and disease. Biomarkers are typically assessed using an enzyme-linked immunosorbent assay (ELISA), which requires the development and manufacture of a specific probe for each analyte. The iterative MS/MS method of the present invention was tested for its ability to profile these known biomarkers in individual patients. Nineteen biomarkers were assayed, using samples from 14 individual patients, as shown in FIG. 7. The method of the present invention was capable of detecting each of the biomarkers and sensitively discerning variations in biomarker levels between individual patients.

Biomarkers for two specific diseases were further investigated. As a representative metabolic disease, nonalcoholic steatohepatitis (NASH) was investigated. NASH is the most severe form of non-alcoholic fatty liver disease (NAFLD). NASH can progress to irreversible cirrhosis, liver failure or liver cancer.

As a representative inflammation-related disease, irritable bowel syndrome (IBS) was also investigated. IBS is a long-term gastrointestinal disorder that can cause persistent discomfort. The causes for IBS remain unclear. Evidence suggests that IBS is associated with prolonged inflammation.

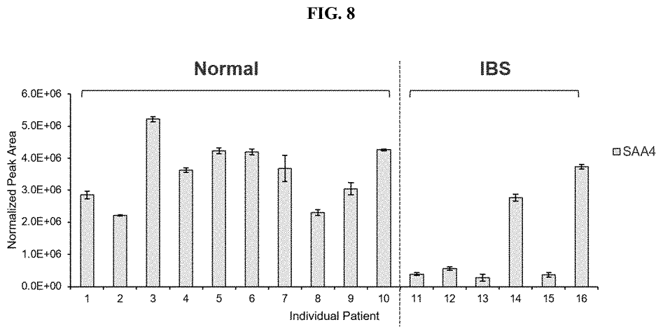

Serum amyloid A (SAA) family proteins are related to inflammation status. Samples from patients with IBS were compared to samples from patients without IBS and levels of SAA4 were quantified using the iterative MS/MS method of the present invention, as shown in FIG. 8. Using this method, lower average expression levels of SAA4 were detected in IBS patients, validating the ability of this method to detect biomarkers indicative of this inflammatory disorder.

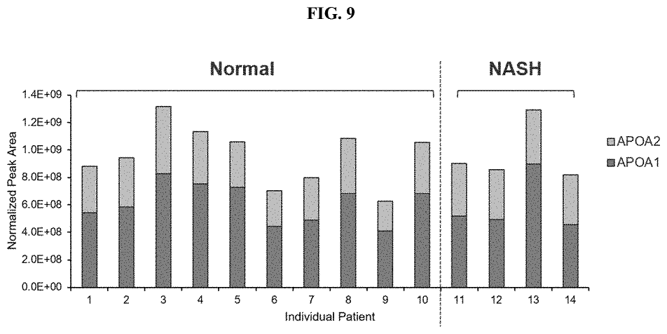

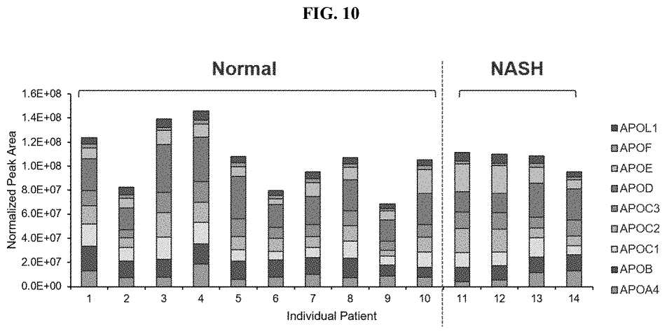

Next, apolipoproteins were selected as potential biomarkers of metabolic disorder. Samples from patients with NASH were compared to samples from patients without NASH and levels of apolipoprotein A-I (apoA-I) and apoA-II, the most abundant apolipoproteins, were compared using the method of the present invention, as shown in FIG. 9. No significant differences between groups were observed. Lower-level apolipoproteins were additionally compared, as shown in FIG. 10. No significant differences between groups were observed. These results indicate that apolipoproteins may not be a useful biomarker for NASH, and demonstrate the ability of the method of the present invention to assay and compare a range of proteins from complex patient samples for biomarker discovery, validation and analysis.

A simple and robust method to identify and quantify plasma proteomics was developed. Minimal plasma sample was required, on the scale of 5 and no plasma depletion was required. Automated iterative MS/MS was employed in a novel application and successfully remedied the dynamic range issue inherent in a complex biological sample such as human plasma. The method was validated for use in assessing patient sample biomarkers in multiple disease states.

What is claimed is:

1. A method for characterizing at least one protein of interest in a biological sample, comprising:
(a) subjecting a biological sample to a chromatography column to obtain a chromatographic elution peak;
(b) performing a tandem mass spectrometry analysis by performing a data-dependent acquisition cycle across the chromatographic elution peak of (a), wherein the cycle includes:
(i) obtaining a mass spectrum scan;
(ii) selecting a plurality of precursor ions from the obtained mass spectrum scan as an automatic exclusion set; and
(iii) obtaining a second mass spectrum scan after excluding the plurality of precursor ions set in the automatic exclusion set; and
(c) characterizing the at least one protein of interest after the acquisition cycle is run for a predetermined number of times.

2. The method of claim 1, wherein said predetermined number of cycles is one, two, three, four, or more cycles.

3. The method of claim 1, wherein a mass error tolerance for selecting a precursor ion for an automatic exclusion set is at about 15 ppm.

4. The method of claim 1, wherein a retention time tolerance for selecting a precursor ion for an automatic exclusion set is from about −0.2 minutes to about +0.4 minutes.

5. The method of claim 1, wherein the automatic exclusion set also includes at least one background ion.

6. The method of claim 1, wherein the automatic exclusion set includes at least one additional precursor ion not from the acquired mass spectrum scan.

7. The method of claim 1, wherein precursor ions from the acquired mass spectrum are not added to the automatic exclusion set if they fall below a predetermined intensity threshold.

8. The method of claim 1, wherein the sample preparation includes direct digestion, optionally wherein direct digestion comprises contacting said sample to trypsin and LysC.

9. The method of claim 1, wherein the chromatography step comprises reverse phase liquid chromatography, ion exchange chromatography, size exclusion chromatography, affinity chromatography, hydrophobic interaction chromatography, hydrophilic interaction chromatography, mixed-mode chromatography, or a combination thereof.

10. The method of claim 1, wherein the mass spectrometer is an electrospray ionization mass spectrometer, nano-electrospray ionization mass spectrometer, or a quadrupole time-of-flight mass spectrometer, wherein the mass spectrometer is coupled to a liquid chromatography system.

11. The method of claim 1, wherein said biological sample is a human sample.

12. The method of claim 1, wherein said biological sample is plasma.

13. The method of claim 1, wherein said at least one protein of interest is a biomarker.

14. A method for characterizing at least one biomarker in human plasma, comprising:
(a) diluting about 5 μL of human plasma in lysis buffer;
(b) taking a sample of said diluted human plasma comprising about 100 μg of plasma protein;
(c) contacting said sample to at least one reduction agent and at least one alkylation agent;
(d) contacting said sample from (c) to trypsin and LysC under digestive conditions to form a digested peptide sample;
(e) subjecting said digested peptide sample to peptide cleanup;
(f) subjecting said digested peptide sample of (e) to an overnight concentration step to form a concentrated peptide sample;
(g) subjecting said concentrated peptide sample to a chromatography column to obtain a chromatographic elution peak;
(h) performing a tandem mass spectrometry analysis by performing a data-dependent acquisition cycle across the chromatographic elution peak of (a), wherein the cycle includes:
(i) obtaining a mass spectrum scan;
(ii) selecting a plurality of precursor ions from the obtained mass spectrum scan as an automatic exclusion set; and
(iii) obtaining a second mass spectrum scan after excluding the plurality of precursor ions set in the automatic exclusion set; and
(c) characterizing the at least one biomarker after the acquisition cycle is run for at least three times.
